(19) United States
(12) Patent Application Publication
(10) Pub. No.: US 2004/0158108 A1
(43) Pub. Date: Aug. 12, 2004

(54) Purification of alcohol

(76) Inventors: Karel A.J. Snoble, Norton Shores, MI (US); Shihan Chen, Palatine, IL (US); Russ Johnson, Elmhurst, IL (US); Stephen F. Yates, Arlington Heights, IL (US); Alexander M. Bershitsky, Glenview, IL (US); Chad D. Garibaldi, Grand Rapids, MI (US)

Correspondence Address: Roberts & Mercanti, L.L.P., P.O. Box 484, Princeton, NJ, 08542-0484, US

(21) Appl. No.: 10/361,349

(22) Filed: Feb. 6, 2003

(51) Int. Cl. C07C 29/143

(52) U.S. Cl. 568/864

(57) ABSTRACT

The invention provides a process for the purification of alcohols, particularly for the purification of isopropyl alcohol. A invention provides a process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; and then recovering a recovered alcohol product from the reaction product.

### BACKGROUND OF THE INVENTION

The present invention relates to the purification of alcohols, or more particularly for the purification of isopropyl alcohol.

It is known in the art to use alcohols, particular isopropyl alcohol, for extraction, in electronic applications as a solvent, as a water removal agent, as well as for analytical applications. In many applications, such as for high performance chromatographs with UV detection and pharmaceutical extraction, very high purity alcohols are required.

Many steps in the semiconductor wafer manufacturing process are followed by a deionized water rinse, which is then followed by a drying step. During this wafer drying step, it is important to prevent watermarks from forming on the surface of the silicon wafers. Watermarks typically form when dissolved contaminants precipitate out of the deionized water as it evaporates from the surface of the wafer. The presence of watermarks on a partially manufactured wafer creates serious difficulties in subsequent manufacturing processes.

Watermark formation on silicon wafers can be minimized or prevented by keeping the deionized water from evaporating off of the wafer surface during the drying process. Several important techniques for achieving this result involve the use of isopropyl alcohol (IPA). In one such technique, the water on the surface of the wafer is displaced by isopropyl alcohol before the water has a chance to evaporate, and then the alcohol is evaporated from the surface of the wafer. Another technique involves condensation of isopropyl alcohol vapor onto the surface of the wafer, causing the water present on the wafer to be taken up by the dry alcohol. The water-rich alcohol then drips off of the wafer before water evaporation can occur, and is replaced by more dry alcohol condensate, which is then evaporated. To minimize or prevent watermarks and to enhance drying, semiconductor manufacturers require ultrapure isopropyl alcohol. Currently, the availability of ultradry and ultrapure isopropyl alcohol from suppliers is limited in relation to the demands of the industry for the chemical. In addition, ultrapure and ultradry isopropyl alcohol purchased from offsite suppliers may lose its purity due to contaminants added during its handling and transportation to the semiconductor manufacturer. Unfortunately, the current methods of purifying isopropyl alcohol are not suited to meet this need. For example, one well-known method of purifying isopropyl alcohol involves simple overhead product distillation. This method, while useful in removing contaminants with boiling points lower than isopropyl alcohol, cannot be used economically to dehydrate isopropyl alcohol to an ultradry level, even though isopropyl alcohol forms a low boiling azeotrope with water. In addition, this method also does nothing to remove those contaminants with boiling points similar to isopropyl alcohol.

In addition, commercially produced bulk alcohols typically contain various amounts of several organic impurities such as acetone, methyl ethyl ketone, as well as other ketone and aldehyde impurities resulting from the synthesis of the alcohol. These ketone and aldehyde impurities are usually present in amounts of a few hundreds of parts per million. For very high purity applications, the impurity levels must be reduced to only a few parts per million. While one may obtain higher purity alcohol forms by distillation processes, it has been determined that ketone and aldehyde impurities are difficult to remove to the required low levels by conventional distillation processes. If these ketone and aldehyde impurities are not removed, alcohols with a UV profile result which are unacceptable for use as a solvent in UV sensitive applications. The resulting purified alcohol product of this process may contain trace amounts of non-alcohol converted products as long as the resulting product has a low UV absorption profile. The present invention provides a process for reducing the amount of ultraviolet light absorbing ketone and/or aldehyde impurities to ultralow levels which have a resulting UV profile which is acceptable in UV sensitive applications.

### DESCRIPTION OF THE INVENTION

The invention provides a process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; and then recovering a recovered alcohol product from the reaction product. The process may be conducted in a batch process, a continuous process or a batch after batch process.

The invention also provides a batch process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; recovering a recovered alcohol product from the reaction product, and optionally discarding a residue of the reaction product.

The invention further provides a batch after batch process wherein after performing the batch process steps above, one subsequently adds additional quantities of a fluid mixture containing an alcohol in addition to ketone impurities and/or aldehyde impurities to a residue of the reaction product obtained after recovery of then recovered alcohol product; causing a further reaction with a sufficient amount of the reducing agent, under conditions wherein the reducing agent is preferentially more reactive with the ketone impurities and/or aldehyde impurities than the alcohol to thereby form a reaction product; and then recovering additional recovered alcohol product from the reaction product.

The invention still further provides a continuous process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; recovering a recovered alcohol product from the reaction product; and then adding additional quantities of a fluid mixture containing an alcohol in addition to ketone impurities and/or aldehyde impurities to a residue of the reaction product obtained after recovery of the recovered alcohol product; causing a further reaction with a sufficient amount of the reducing agent, under conditions wherein the reducing agent is preferentially more reactive with the ketone impurities and/or aldehyde impurities than the alcohol to thereby form a reaction product; and then recovering additional recovered alcohol product from the reaction product.

The production of alcohols is well known in the art and such are generally commercially available, for example from Aldrich, of Milwaukee, Wis.

The purification technique of this invention may be applied to alcohols which are or can be put into a fluid form. Such include C₁ to C₁₂ alcohols, particularly, C₁ to C₁₂ aliphatic alcohol, more particularly C₁ to C₆ aliphatic alcohols such as methyl alcohol, ethyl alcohol, propyl alcohols, butyl alcohols, pentyl alcohols and hexyl alcohols. The process is most particularly appropriate for the purification of isopropyl alcohol.

The purification is conducted by first contacting a fluid alcohol containing mixture with a reducing agent. Suitable reducing agents are capable of transferring hydrogen atoms to the ketone impurities and/or aldehyde impurities and thus reduce the ketone impurities and/or aldehyde impurities to alcohols. Useful reducing agents include borohydrides, hydrides, boranes, and combinations thereof among others. Preferred borohydrides include metal borohydrides such as sodium borohydride, lithium borohydride, potassium borohydride, and cesium borohydride, metal borohydrides in the presence of metal salts, such as sodium borohydride in the presence of CoCl₂, NiCl₂, or SnCl₂; zinc borohydride, alkoxy borohydrides such as KBH(OCH(CH₃)₂)₃, acetoxyborohydrides such as sodium triacetoxyborohydride (NaBH(OCOCH₃)₃), cyanoborohydrides, quaternary ammonium salt borohydrides, for example, (n-Bu)₄BH₄, and trialkylborohydrides, for example K(sec-Bu)₃BH. Useful hydrides include aluminum hydride, lithium aluminum hydride, sodium aluminum hydride, and LiAIH(OCH(CH₃)₂)₃. Useful boranes include borane, borane complex with triethylamine, and borane complex with triphenylphosphine. Other useful reducing agents include Raney Nickel. The preferred reducing agents are the alkali metal borohydrides, and sodium borohydride is particularly convenient because of its effectiveness and ready availability. In a preferred embodiment, the reducing agent is dispersed in the alcohol fluid mixture. Preferably the reaction is conducted in the temperature range from about −20° C. to about 200° C., preferably from about 15° C. to about 120° C. and more preferably from about 15° C. to the normal boiling point of the alcohol. Preferably the reducing agent is dispersed in the fluid mixture in an amount such that the reducing agent provides at least one hydrogen atom per molecule of the ketone and/or aldehyde impurities in the alcohol mixture. Usually the reducing agent is present in an excess of the amount required to react with the ketone and/or aldehyde impurities in the alcohol mixture. In an alternate embodiment, the reaction is conducted by contacting the alcohol fluid mixture with the reducing agent wherein the reducing agent is immobilized on a support such as a borane polymerically bound with polystyrene or sodium borohydride held within the pores of a zeolite which is alkaline stable or sodium borohydride in combination with an anion exchange resin.

Then a recovered alcohol product is recovered from the reaction product, preferably by distillation. Distillation may be conducted by heating the reaction product in a distillation apparatus at a temperature above the boiling point of the alcohol. The recovered alcohol product contains about 100 ppm or less of ketone impurities and/or aldehyde impurities, preferably about 10 ppm or less of ketone impurities and/or aldehyde impurities, and more preferably about 1 ppm or less of such impurities. The amount of such impurities may be determined by the UV of the recovered alcohol product.

Preferably the recovered alcohol product has an ultraviolet absorbance in a 5 cm UV cell of about 0.8000 or less at 225 nm, an ultraviolet absorbance of about 0.1000 or less at 250 nm, an ultraviolet absorbance of about 0.0250 or less at 300 nm, and an ultraviolet absorbance of about 0.0250 or less at 400 nm.

The process may be conducted in a batch process, a continuous process or a sequential batch after batch process. In a batch process, the steps above are followed and thereafter the reaction vessel may be emptied and cleaned prior to conducting the process again. In a continuous process, one subsequently adds additional quantities of the fluid mixture containing an alcohol in addition to ketone impurities and/or aldehyde impurities to a residue of the reaction product obtained after recovery of the recovered alcohol product. This causes a further reaction with a sufficient amount of the reducing agent, under conditions wherein the reducing agent is preferentially more reactive with the ketone impurities and/or aldehyde impurities than the alcohol to thereby form a reaction product; and then recovering additional recovered alcohol product from the reaction product. The process continues with additional continuous flows of the alcohol containing fluid mixture into the vessel with optional additions of reducing agent. In a batch after batch process, further amounts of alcohol containing fluid mixture, and optionally reducing agent, are added in a batchwise fashion into the reaction vessel containing a residue of the reaction product with further purified alcohol recovery.

The following non-limiting examples serve to illustrate the invention.

### EXAMPLE 1

Reacting Isopropyl Alcohol with Sodium Borohydride to Improve Isopropyl Alcohol UV Characteristics

To a clean and dry IL distillation flask add 200 ml raw material isopropyl alcohol (IPA). Using a plastic funnel, 24 mg of sodium borohydride (NaBH₄, 98% purity) powder is added. The funnel is rinsed with 100 ml of IPA to ensure all the NaBH₄ is added to the distillation flask. The flask now contains 24 mg (100 ppm) NaBH4 and 300 ml IPA. The flask is attached to a clean and dry distillation column of about containing about 60 cm height of stainless steel expanded metal fractionating medium. The flask is heated to reflux over the time indicated and refluxed for 20 minutes. A sample of 20 ml overhead IPA is then collected (reflux ratio of 3:17) after which the main fraction is collected, in a clean and dry bottle, at a reflux ratio of 17:3. The distillation is stopped when 20-30 ml of IPA remains in the distillation flask. The main fraction is analyzed by UV spectroscopy in a 5 cm UC cell.

A 2×2 full factorial experiment using five replicates was conducted with raw material quality and distillation flask heat-up time being the variables. Sodium borohydride was added to achieve 100 ppm concentration in all cases. UV absorption was measured at four wavelengths (225, 250, 300, and 400 nm). The results in the following table show that suitable quality, with respect to UV absorption, was obtained in all cases.

Five Cm UV Cell Results from an Average of Five
Data Points for Each Design of Experiment (DOE)
Entry, NaBH₄ at 100 ppm Concentration

| | Wavelength | | | | | UV cutoff |
|---|---|---|---|---|---|---|
| Sample Description | 225 nm | 250 nm | 300 nm | 400 nm | Pass/Fail | Nm |
| Finished product | <0.8 | <0.1 | <0.025 | <0.025 | Pass | NA |
| Target UV Absorption | | | | | | |
| Specification | | | | | | |
| Feedstock A | 0.7443 | 0.1098 | 0.0268 | 0.0001 | Fail | 220.2 |
| Feedstock A, distilled | 0.7111 | 0.1065 | 0.0202 | 0.0007 | Fail | 219.6 |
| Feedstock B | 0.6889 | 0.1106 | 0.0302 | 0.0004 | Fail | 221.1 |
| Feedstock B, distilled | 0.7185 | 0.0924 | 0.0172 | −0.0003 | Pass | 219.8 |
| DOE IPA Feedstock A, | 0.4175 | 0.0520 | 0.0021 | −0.0001 | Pass | 213.1 |
| distilled with NaBH₄ | | | | | | |
| using 2 hr 10 min heat | | | | | | |
| time | | | | | | |
| DOE IPA Feedstock B, | 0.3773 | 0.0467 | 0.0011 | 0.0002 | Pass | 212.1 |
| distilled with NaBH₄, | | | | | | |
| using 2 hr, 10 min heat | | | | | | |
| time | | | | | | |
| DOE IPA Feedstock A, | 0.3848 | 0.0461 | 0.0054 | −0.0006 | Pass | 211.7 |
| distilled with NaBH₄ | | | | | | |
| using 15 min heat time | | | | | | |
| DOE IPA Feedstock B, | 0.3850 | 0.0503 | 0.0040 | 0.0000 | Pass | 212.2 |
| distilled with NaBH₄ | | | | | | |
| using 15 min heat time | | | | | | |

### EXAMPLE 2

Effect of NaBH₄ Levels on IPA Product UV Characteristics

The procedure described above for treating IPA with NaBH₄ to improve UV characteristics was followed, with the exception that the level of NaBH₄ was varied. The raw material used was Feedstock B, and the heat time was 15 minutes. The results in the table below show that IPA can be beneficially treated with levels of NaBH₄ ranging from 50 to 1000 ppm by weight. One sees that the higher levels of NaBH₄ result in measurably better 5 cm cell UV absorption than do lower levels, but all treatment levels result in suitable quality material.

Effects of Various Sodium Borohydride Treatment Levels
on IPA 5 cm UV Cell Absorption Characteristics

| Wavelength | 1000 ppm | 100 ppm | 50 ppm | 0 ppm | USL* |
|---|---|---|---|---|---|
| 225 | 0.0779 | 0.1134 | 0.1445 | 0.6027 | 0.8 |
| 250 | 0.0081 | 0.0177 | 0.0241 | 0.0795 | 0.1 |
| 300 | 0.0013 | 0.0074 | 0.0109 | 0.0155 | 0.025 |
| 400 | 0.0017 | 0.0012 | 0.0008 | | 0.025 |

Note:
0 ppm is finished product IPA data mean. Samples are kept under inert gas.
*Upper specification limit

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; and then recovering a recovered alcohol product from the reaction product.

2. The process of claim 1 wherein the alcohol comprises a C₁ to C₁₂ alcohol.

3. The process of claim 1 wherein the alcohol comprises a C₁ to C₁₂ aliphatic alcohol.

4. The process of claim 1 wherein the alcohol comprises a C₁ to C₆ aliphatic alcohol.

5. The process of claim 1 wherein the alcohol comprises isopropyl alcohol.

6. The process of claim 1 wherein the reducing agent is capable of transferring hydrogen atoms to the ketone impurities and/or aldehyde impurities.

7. The process of claim 1 wherein the reducing agent is capable of reducing the ketone impurities and/or aldehyde impurities to alcohols.

8. The process of claim 1 wherein the reducing agent comprises one or more materials selected from the group consisting of hydrides, borohydrides, boranes and combinations thereof.

9. The process of claim 1 wherein the reducing agent comprises one or more materials selected from the group consisting of metal borohydrides, metal borohydrides in the presence of metal salts, alkoxy borohydrides, acetoxyborohydrides, cyanoborohydrides, quaternary ammonium salt borohydrides, and trialkylborohydrides.

10. The process of claim 1 wherein the reducing agent comprises one or more materials selected from the group consisting of sodium borohydride, lithium borohydride, potassium borohydride, cesium borohydride, sodium borohydride in the presence of CoCl₂, NiCl₂, or SnCl₂; zinc borohydride, sodium triacetoxyborohydride, KBH(OCH(CH₃)₂)₃, (n-Bu)₄BH₄, K(sec-Bu)₃BH, aluminum hydride, lithium aluminum hydride, sodium aluminum hydride, LiAIH(OCH(CH₃)₂)₃, borane, borane complex with triethylamine, borane complex with triphenylphosphine, and Raney Nickel.

11. The process of claim I wherein the reducing agent comprises sodium borohydride.

12. The process of claim 1 wherein the reaction is conducted by heating the fluid mixture to reflux.

13. The process of claim I wherein the reaction is conducted at a temperature of from about −20° C. to about 200° C.

14. The process of claim 1 wherein the reaction is conducted at a temperature of from about 15° C. to about 120° C.

15. The process of claim 1 wherein the reaction is conducted at a temperature of from about 15° C. to the normal boiling point of the alcohol.

16. The process of claim 1 wherein the reaction is conducted under alkaline conditions.

17. The process of claim 1 wherein the reducing agent is dispersed in the fluid mixture.

18. The process of claim 1 wherein the reducing agent is dispersed in the fluid mixture, and wherein the amount of reducing agent is such that the reducing agent provides at least one hydrogen atom per molecule of the ketone impurities and/or aldehyde impurities.

19. The process of claim 1 wherein the reaction is conducted by contacting the fluid mixture with the reducing agent wherein the reducing agent is immobilized on a support.

20. The process of claim 1 wherein the recovered alcohol product is recovered from the reaction product by distillation.

21. The process of claim 1 wherein the recovered alcohol product has an ultraviolet absorbance in a 5 cm UV cell of about 0.8000 or less at 225 nm, an ultraviolet absorbance of about 0.1000 or less at 250 nm, an ultraviolet absorbance of about 0.0250 or less at 300 nm, and an ultraviolet absorbance of about 0.0250 or less at 400 nm.

22. The process of claim 1 wherein the recovered alcohol product contains about 100 ppm or less of ketone impurities and/or aldehyde impurities.

23. The process of claim 1 wherein the alcohol comprises isopropyl alcohol; the reducing agent comprises sodium borohydride which is dispersed in the fluid mixture in an amount to provide at least one hydrogen atom per molecule of the ketone impurities and/or aldehyde impurities; the reaction is conducted under alkaline conditions and by heating the fluid mixture to reflux; the recovered alcohol product is recovered from the reaction product by distillation; and wherein the recovered alcohol product has an ultraviolet absorbance in a 5 cm UV cell of about 0.8000 or less at 225 nm, an ultraviolet absorbance of about 0.1000 or less at 250 nm, an ultraviolet absorbance of about 0.0250 or less at 300 nm, and an ultraviolet absorbance of about 0.0250 or less at 400 nm.

24. The process of claim 23 wherein the recovered alcohol product contains about 100 ppm or less of ketone impurities and/or aldehyde impurities.

25. A continuous process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; recovering a recovered alcohol product from the reaction product; and then adding additional quantities of a fluid mixture containing an alcohol in addition to ketone impurities and/or aldehyde impurities to a residue of the reaction product obtained after recovery of the recovered alcohol product; causing a further reaction with a sufficient amount of the reducing agent, under conditions wherein the reducing agent is preferentially more reactive with the ketone impurities and/or aldehyde impurities than the alcohol to thereby form a reaction product; and then recovering additional recovered alcohol product from the reaction product.

26. A batch process for reducing the amount of ultraviolet light absorbing ketone impurities and/or aldehyde impurities in a fluid mixture containing an alcohol in addition to ketone impurities and/or aldehyde impurities, which comprises reacting a fluid mixture containing an alcohol in addition to ketone impurities and/or aldehyde impurities, with a sufficient amount of a reducing agent, under conditions wherein the reducing agent is preferentially more reactive with the ketone impurities and/or aldehyde impurities than the alcohol to thereby form a reaction product; recovering a recovered alcohol product from the reaction product, and optionally discarding a residue of the reaction product.

27. The process of claim 26 further comprising subsequently adding additional quantities of a fluid mixture containing an alcohol in addition to ketone impurities and/or aldehyde impurities to a residue of the reaction product obtained after recovery of then recovered alcohol product; causing a further reaction with a sufficient amount of the reducing agent, under conditions wherein the reducing agent is preferentially more reactive with the ketone impurities and/or aldehyde impurities than the alcohol to thereby form a reaction product; and then recovering additional recovered alcohol product from the reaction product.
